(19) United States
(12) Patent Application Publication
(10) Pub. No.: US 2009/0024171 A1
(43) Pub. Date: Jan. 22, 2009

(54) Anatomical Anterior Vertebral Plating System

(76) Inventor: Vincent Leone, Manhasset, NY (US)

Correspondence Address: GORDON & JACOBSON, P.C., 60 LONG RIDGE ROAD, SUITE 407, STAMFORD, CT, 06902, US

(21) Appl. No.: 11/779,941

(22) Filed: Jul. 19, 2007

(51) Int. Cl. A61B 17/70; A61B 17/56; A61B 17/58

(52) U.S. Cl. 606/280

(57) ABSTRACT

An anterior vertebral plate, systems utilizing such plate, and methods of implanting such plates and systems are provided. The plate is anatomically contoured to fit the vertebral anatomy. The plate may include anatomical curves to facilitate seating on the anterior vertebral surface and a concave recess for accommodating segmental vessels.

### BACKGROUND OF THE INVENTION

1. Field of the Invention

This invention relates broadly to surgical devices and methods. More particularly, this invention relates to orthopedic plates for implantation on the anterior aspect of vertebrae.

2. State of the Art

Whether due to injury and damage conditions such as fracture and disc herniations, or tumors, infection, degeneration, or aging, problems with the spine are increasing. As a result, orthopedic intervention at the spine is more common.

The approach to attack the compressive pathology at each level (i.e., a disc or vertebrae) in the thoracolumbar region is relatively consistent. If the pathology is limited to a disc, the disc is removed, a disc spacer is provided at the level of the removed disc, and support is provided across the vertebrae on either side of the disc spacer, using e.g., plates and rods coupled to the plates. If the pathology includes a body of a vertebra, the body of the vertebra is removed, intervening discs are removed, appropriate spacers installed, and the necessary support implanted.

By way of example, the Kaneda System, described in U.S. Pat. No. 5,603,714, includes steel plates that are coupled to vertebrae with bone screws and nuts. Supporting rods are coupled to the screws to provide longitudinal stability and transverse fixation elements are provided to stabilize the rods over the disc spacer. The support system is assembled in situ. The Kaneda System allows correction of deformities and provides sufficient rigidity for stabilizing spinal fractures and recovering from a resection spinal tumor operation.

The approach for using the Kaneda system or one similar thereto is generally open surgery with a lateral approach to provide access to the anterior spine. The anterior spine is that portion anterior of the spinal cord. The anterior spine permits purchase of vertebral support plates and screws in a manner which does not interfere with the spinal cord.

Recently there has been a great push to adapt surgical techniques and systems to permit a minimally invasive approach to spinal surgery. Minimally invasive approaches, where possible, reduce patient trauma. However, any minimally invasive approach should still be suitable for different patients and easily practiced by a surgeon in various situations.

### SUMMARY OF THE INVENTION

The invention includes a spinal fixation system for anterior thoracolumbar fixation including one or more anterior spinal plates, and methods of spinal fixation.

One embodiment of an anterior spinal fixation system includes a plurality of anterior spinal plates, bone screws, rods and optionally cross connectors. The spinal plates are each sized to fit on an anterior lateral portion of respective vertebrae. In one embodiment, and accord with a preferred aspect of the invention, each spinal plate is anatomically contoured to fit the anatomy of a single vertebra, with (i) a concavity being provided to the plate in the anterior-posterior dimension, and (ii) a convexity provided between peripheral portions of the plate, such convexity extending within the cephalo-caudal or superior-inferior dimension. Further, (iii) a preferably concave recess is provided at a central portion in the cephalo-caudal dimension of the plate and running across the plate in the anterior-posterior dimension. In a preferred embodiment, and not by limitation, the concave recess is generally a mirror image of the bone shape at the location underlying the recess. The plate also preferably includes spikes at a lower surface for initial fixation of the plate on the vertebral body and bone screw holes for receiving the bone screws. Plates may be provided with different bone screw holes locations depending upon whether the plate is for relatively cephalo or caudal placement.

Each bone screw has a shaft provided with a bone engaging thread and a head including a slot oriented transverse to the shaft. Two rods are receivable within the slots of the heads of two spaced apart sets of bone screws coupled in two or more plates. Set screws or other means are provided to fix the rods relative to the screw heads. Cross connectors may also be provided to stabilize central portions of the rods together between the locations of the screw heads.

The elements of the system facilitate a minimally invasive approach during surgery. Each of the elements can be passed through one or more ports inserted into the thoracic cavity and then assembled in situ so that an open surgical procedure is unnecessary. Further, the contour at the lower (bone contacting) surface of the plates, namely the concavity in the anterior-posterior dimension and the convexity at the superior and inferior peripheral portions of each plate generally correspond to the anatomy. As such, the plates will self-guide to the cephalo-caudal center of the respective vertebrae. Moreover, the segmental vessels which extend across the surface of the body of the vertebrae and through the neural foramen to feed the anterior spinal artery which provides blood to the spinal cord will not be damaged during the procedure. The concave recess defines a space which offsets the lower surface of the plate relative to the segmental vessels extending in an anterior-posterior direction over a central portion of the surface of each vertebrae. Such offset prevents or limits contact of the plate against the segmental vessels and thereby ensures that the segmental vessels remain intact and unobstructed, obviating the need for a time consuming vascular dissection that would otherwise be required and which may otherwise prevent or limit a minimally invasive, through-port approach.

In accord with another embodiment of the invention, a rigid bridge element is fixedly coupled between two plates and assumes the function of the rods and cross connectors. The bridge element may be coupled to the plates with bone screws, set screws or other elements. When the bridge element couples two plates together, each plate is anatomically contoured, as described above, and includes a concave recess at a central portion thereof (i.e., transverse to the cephalo-caudal axis) and running across the entirety of the plate to define a space between the plate and bone which offsets the lower surface of the plate relative to the segmental vessels. Such offset prevents or limits contact of the plate against the vessels and thereby ensures that the segmental vessels remain intact and unobstructed.

In accord with a further embodiment of the invention, a unitary anterior spinal plate is sized to extend across at least one vertebral level (i.e., at least from one vertebra across a disc space and to another vertebra) of the spine. At each level at which the plate is to be attached to the anterior portion of the spine, the plate includes (i) a concavity in the anterior-posterior dimension, (ii) a convexity between peripheral portions of each respective vertebral level of the plate, such curve extending within the cephalo-caudal (superior-inferior or SI) dimension, and (iii) a concave recess at a central portion of each vertebral level in the cephalo-caudal dimension and running transverse to the plate in the anterior-posterior dimension. Each recess defines a space between the plate and bone which offsets the lower surface of the plate relative to the segmental vessels. Such offset prevents or limits contact of the plate against the vessels and thereby ensures that the segmental vessels remain intact and unobstructed.

Additional advantages of the invention will become apparent to those skilled in the art upon reference to the detailed description taken in conjunction with the provided figures.

### BRIEF DESCRIPTION OF THE DRAWINGS

FIG. 1 is a top view of an anterior vertebral plate according to the invention.

FIG. 2 is a bottom view of the plate of FIG. 1.

FIG. 3 is an anterior view of the plate of FIG. 1.

FIG. 4 is a superior view of the plate of FIG. 1

FIG. 5 is a perspective view of the plate of FIG. 1.

FIG. 6 is a section view of an alternate plate embodiment.

FIG. 7 is a section view of another alternate plate embodiment.

FIG. 8 is a section view of a yet another alternate plate embodiment.

FIG. 9 is a section view of a still another alternate plate embodiment.

FIG. 10 illustrates a plurality of thoracolumbar vertebrae with the segmental vessels extending in a medial-lateral direction over the vertebrae.

FIG. 11 illustrates an anterior bone plate system according to the invention attached to thoracolumbar vertebrae.

FIG. 12 is a partial section view across line 12-12 in FIG. 11.

FIG. 13 is longitudinal section view across an exemplar bone screw, rod and set screw system for use with a plate according to the invention.

FIG. 14 is a second embodiment of an anterior bone plate system according to the invention.

FIG. 15 is a third embodiment of an anterior bone plate system according to the invention.

FIG. 16 is a fourth embodiment of an anterior bone plate system according to the invention.

### DETAILED DESCRIPTION

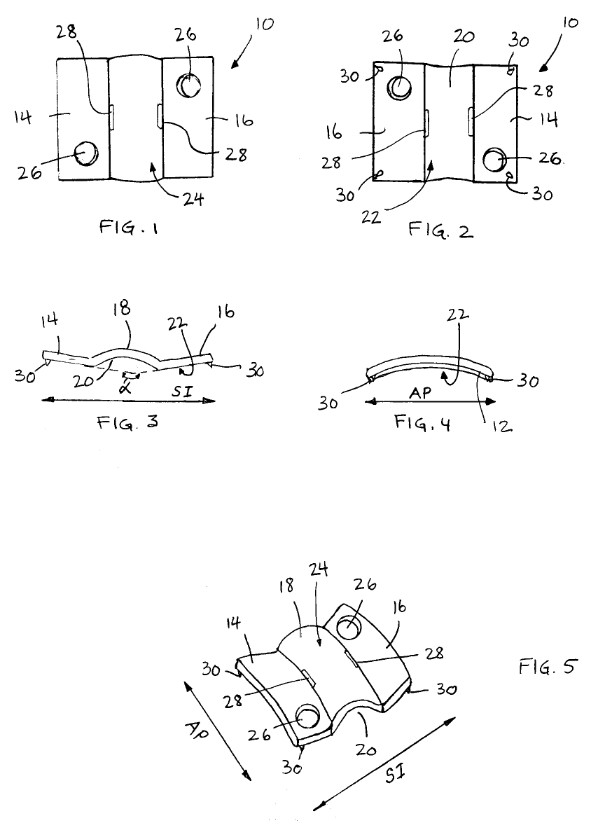

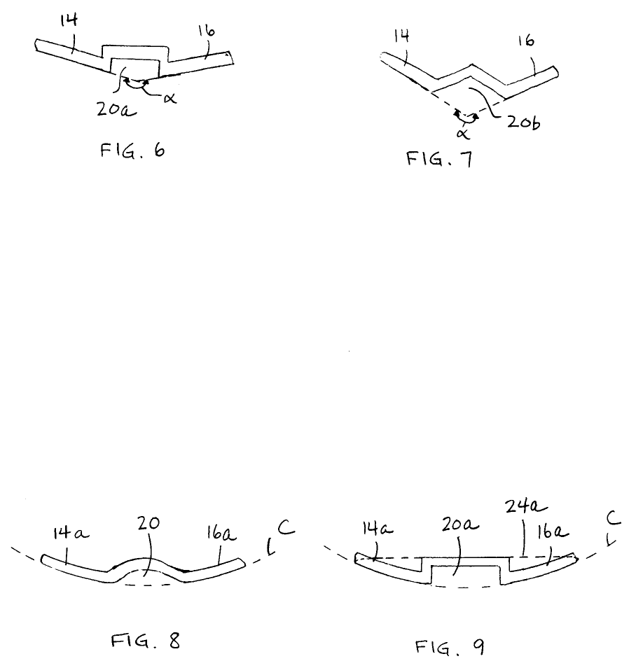

Turning now to FIGS. 1 through 5, an anterior spinal fixation plate 10 is shown. In accord with a preferred aspect of the invention, each plate is anatomically contoured to fit the anatomy of a single vertebra. As such, with respect to the anatomy and the dimensional axes shown, each plate preferably includes a concavity curve 12 provided to the plate in the anterior-posterior (AP) dimension (as shown in FIG. 4). The plate is also preferably formed with a convexity; i.e., a convex relationship provided between the peripheral superior and inferior portions 14, 16 of the plate (FIG. 3); i.e., the lower bone contacting surfaces of portions 14, 16 are out-of-plane in the cephalo-caudal or superior-inferior (SI) dimension. For purposes of the disclosure, a convex relationship includes the peripheral portions 14, 16 angled relative to each other, where angle α is between 180° and 240°, and more preferably where α is between 180° and 220°. Such angled relationship of peripheral portions 14, 16 is shown in the embodiments of FIGS. 3, 6 and 7. The preferred convexity will better accommodate the plate to the surface of the bone. For purposes of the disclosure, a convex relationship also includes the peripheral portions 14a, 16a lying along a preferably common, preferably smooth curve C, as shown in the plate embodiments of FIGS. 8 and 9. The described convex and concave curves cause the plate to seat closely against the anterior surface of the vertebra. In addition, a recess 20 is provided to the plate between the superior and inferior portions 14, 16. More particularly, recess 20 is provided at a central portion 18 of the plate in the SI dimension and running across the plate in the AP dimension (FIGS. 1-3 and 5). Such recess 20 is preferably a smooth curve concave recess as shown in FIGS. 3, 5 and 8; the recess may also be formed as an angular channel, e.g., a rectangular channel 20a (FIGS. 6 and 9) or an angled arch 20b (FIG. 7). In a preferred embodiment, and not by limitation, the recess 20 is generally a mirror image of the concave bone shape at the location underlying the recess. As described in more detail below, this provides a small recess with sufficient space for the bone contacting surface of the plate to be offset from segmental vessels that extend across the vertebral surface so that the plate can be placed on the vertebra without first dissecting the segmental vessels, as described with respect to FIG. 12 below.

While it is preferred that the plate 10 have the three features described above: (i) a concavity in the AP dimension, (ii) a convex relationship provided between the peripheral portions in the cephalo-caudal (or SI) dimension, and (iii) a recess in the SI dimension and running across the plate in the AP dimension, it is also within the scope of the invention for the plate to include a subset of these features.

While the plate 10 is preferably formed with matching lower bone contacting surface 22 and upper surface 24, both provided with the above described anatomical contour, it is recognized that the plate may be formed so that only the lower bone contacting surface 22 includes the described anatomical contour, with the upper surface forming any other shape. However, if the upper surface has a shape different from the lower surface, it is preferable that the upper surface have a shape adapted to minimize surrounding tissue irritation. By way of example, the upper surface 24a may be planar, as shown in broken line in FIG. 9.

The plate 10 also includes screw holes 26 for receiving screws therein. The placement of the screw holes may be uniform for all plates; however in a preferred embodiment plates intended for cephalad vertebral placement have a different screw hole placement than plates designed for caudal placement, with such placement preferably having linear symmetry. See, e.g., plates 10a, 10b in FIG. 11, discussed below. The screw holes 26 may be unthreaded or threaded and adapted to receive polyaxial screws, fixed angle bone screws that have an unthreaded or threaded shaft portion, or fixed angle bone screws that have a threaded or unthreaded head portion to secure the bone screws to the plate. Further, the plate may be provided with slots 28 at which the plate can be engaged by a tool to facilitate maneuvering of the plate through a port, seating of the plate on the bone, etc. Optionally small spikes 30 may be provided to the lower bone contacting surface 32. The spikes 30 impart an anti-glide feature to the plate to prevent inadvertent movement of the plate on the vertebral surface.

In accord with a first embodiment, the plate 10 is sized to fit on an anterior lateral portion of a single thoracolumbar vertebra. The plate 10 preferably has a length in the cephalo-caudal (SI) dimension between 25 mm to 40 mm, and a width in the AP dimension between 30 mm to 45 mm. The recess 20 preferably has a height relative to the bone of 1-3 mm, a minimum width of 3-6 mm, and a maximum width of 5-10 mm. It is also preferable that the width of the recess (cephalo-caudad (SI) dimension) be approximately one third the total length (SI dimension) of the plate. It is appreciated that the plate may be provided in several discrete sizes, e.g., small, medium and large, to accommodate human individuals in the range of, e.g., 40 to 120 kgs. The plate is made of metal, or another suitably rigid biocompatible material. Preferred materials include stainless steel and titanium.

Turning now to FIG. 10, the right lateral side of a portion of the thoracolumbar spine 50 including two vertebrae 52, 54 and a disc 56 are shown. With respect to vertebra 52, each vertebra includes a vertebral body 58, a vertebral foramen 60, and various processes 64 that connect adjacent vertebra together. The vertebral foramen 60 of multiple vertebrae together define the spinal canal 66 which contains the spinal cord 68, as well the meninges, the nerve roots and blood vessels. Pairs of segmental blood vessels 70 extend in a medial-lateral direction across the anterior surface 72 of vertebrae 52, 54 and enter the spinal canal through the neural foramen 74 defined at the intersection of the adjacent vertebra. The segmental vessels 70 are responsible for feeding blood the anterior spinal artery. If the segmental vessels 70 are obstructed, the result can be an anterior spinal cord infarct, resulting in paralysis.

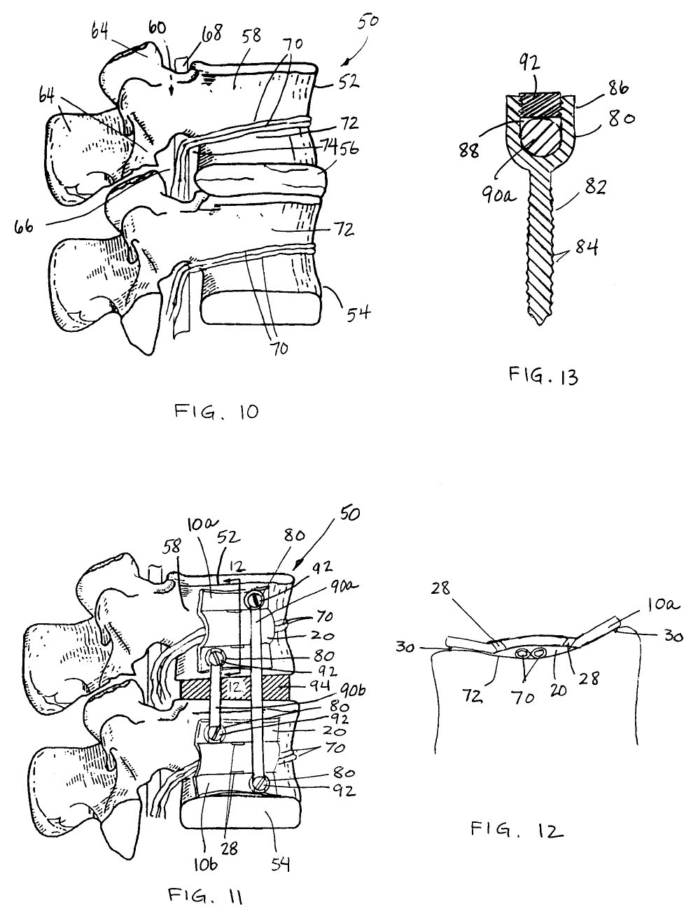

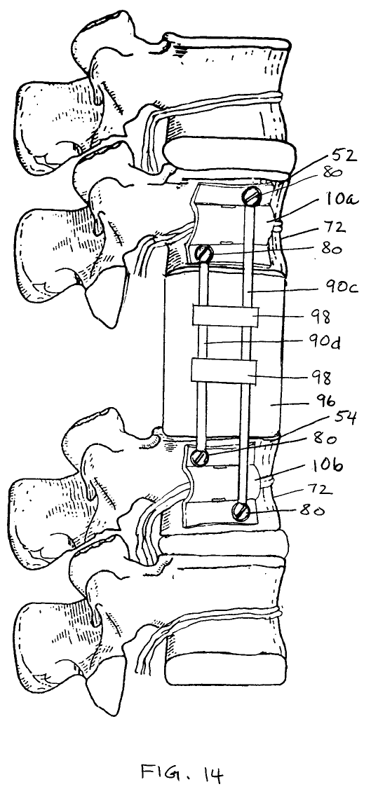

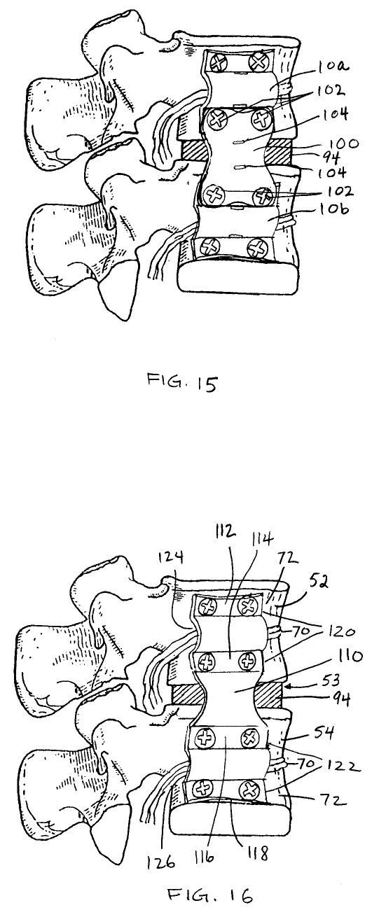

Referring now to FIGS. 11 through 13, the plate of the invention is preferably used as part of an anterior spinal fixation system on the thoracolumbar portion of the spine 50, such system including a plurality of anterior spinal plates, screws, rods and optionally cross connectors, as described as follows. The system may be implanted in an open procedure or a closed minimally invasive procedure. Regardless of which procedure is used, the approach is similar; however, it will become apparent from the following that the plate facilitates a minimally invasive approach if desired.

The patient's spine is approached by placing the patient in a preferably lateral decubitus position. The patient is then either opened with appropriate incision to provide access to the vertebral level requiring treatment, or, using known methodology, thoracoscopic ports are inserted into the patient providing the requisite access for tools and implants. As described below, the construct of the system facilitates a minimally invasive approach during surgery, as each of the elements of the system can be passed through one or more ports, with the system assembled in situ.

For disc replacement, the disc material is removed. The vertebral body is measured, and the appropriate size of plate is selected based upon the measurement. The plate 10a is then maneuvered to the rostral side of the site and positioned on the lateral anterior surface 72 of a vertebra 52. The plate may be maneuvered by engagement of an appropriate tool within slots 28 or by other structural engagement. In addition, means other than slots, e.g., threads or recesses, may be provided to the plate for releasable engagement by a tool for such maneuvering to the implant site. The anatomical contour of the plate 10a causes it to naturally assume a proper location on the anatomy of the vertebra, and the short spikes 30, if provided to the lower surface of the plate, prevent inadvertent movement on the anterior surface of the vertebra. It is particularly noted that in a minimally invasive surgical approach the plate contour is practically self-seating. Furthermore, as shown in FIG. 12, the recess 20 at the lower surface of the plate provides clearance for the segmental vessels 70. Here it is seen how a substantially mirror image shape to the underlying surface of the vertebra provides adequate clearance (with the recess and underlying bone defining a conduit for the vessels) such that the segmental vessels do not need to be dissected, which is otherwise required, to ensure that the vessels are not obstructed or otherwise damaged. Each of these features greatly facilitates a minimally invasive approach, and together offer significant advantage over the prior art.

Once the plate 10a is on the anterior surface 72 of the vertebra 52, holes are drilled into the cephalad vertebral body 58 through the screw holes 26, and screws 80 are inserted through the screw holes and drilled holes to engage the vertebral body. The process is repeated for a second plate 10b positioned on the anterior surface of caudal vertebra 54, with holes drilled into vertebral body through the screws holes, and screws 80 are inserted through the screw holes and drilled holes to engage the vertebral body. As indicated above, plate 10b has a screw holes located in different locations from plate 10a. Each screw 80 has a shaft 82 provided with a bone engaging thread 84 and a head 86 including an slot 88 for receiving a rod 90a oriented transverse to the shaft 82 and parallel to the spinal column, and a threaded opening for receiving a set screw 92.

After the plates 10a, 10b are anchored to the vertebral bodies, the disc replacement device 94 is inserted between the cephalad and caudal vertebrae on which the plates 10a, 10b are implanted. The disc replacement device 94 may be autologous bone, allograft bone, synthetic bone, or other synthetic replacement device. The rods 90a, 90b are then inserted into the slots 88 in the bone screws 80, and the set screws 92 are inserted into the threaded upper portions of slots 88 in the bone screws 80 and tightened to secure the rods 90a, 90b in position. The rods 90a, 90b may be of different length, with a shorter rods 90b preferably provided in a relatively posterior position.

In addition, other bone screws (fixed angle and polyaxial), rods, set screws, or other means for fixing rods or other supports relative to the plates, as described in U.S. Pat. Nos. 5,364,399 to Lowery; 5,498,263 to DiNello; 5,603,714 to Kaneda; 5,899,904 to Errico; 6,454,771 to Michelson; and 6,136,002 to Shih, which are all hereby incorporated by reference herein, may be used in combination with the described plates.

Turning now to FIG. 14, another embodiment of a system according to the invention is shown. The system is provided for use when the pathology spans across a vertebral level including a vertebra. Appropriate disc and end plate material cephalad and caudal to the damaged vertebral body is removed. A corpectomy is then performed. The plates 10 are implanted with bone screws 80, as discussed above, on the anterior lateral surfaces 72 of vertebrae 52, 54 rostral and caudal of the corpectomy site. A vertebral body implant device 96 is then implanted at the corpectomy site. Rods 90c, 90d are then positioned within the slots 88 of the bone screws to provide a stabilizing structure about the implant device 96. It is appreciated that rods 90c, 90d are longer than rods 90a, 90b. In view of such longer length, cross connectors 98 may also be provided to stabilize central portions of the rods 90c, 90d together between the locations of the screw heads.

Referring now to FIG. 15, in accord with another embodiment of the system of the invention, (shown with respect to a diskectomy but suitable for use during a corpectomy), a diskectomy is performed, two plates 10a, 10b are implanted on the vertebrae 52, 54 cephalad and caudal of the diskectomy, and a disc replacement device 94 is inserted at the diskectomy location. A rigid bridge element 100 is then rigidly coupled between the two plates 10a, 10b. In the embodiment shown the bridge is sized to extend across one level, a disc; however, it is appreciated that the bridge 100 may be longer in length for use in a corpectomy. The bridge element 100 may be coupled to the plates 10a, 10b with the bone screws 102 that extend through the bridge elements 100 and plates 10a, 10b, or with other coupling structure, e.g., set screws. The bridge element 100 may also include slots 104 or other structure permitting the bridge to be releasably engaged by a tool that facilitates maneuvering the bridge, particularly through a port. The bridge element 100 may have a shape other than shown, e.g., be narrower or wider than illustrated or of different thickness than plates 10a, 10b, etc. The bridge element 100 assumes the function of the rods and cross connectors of prior embodiments.

The two plates 10a, 10b of the assembled construct each includes a concave recess 20 at a central portion thereof in the cephalo-caudal (SI) dimension and running across the plate in the AP dimension to defines a space between the plate and bone which offsets the lower surface of the plate relative to the segmental vessels. Such offset prevents or limits contact of the plate against the vessels and thereby ensures that the segmental vessels remain intact and unobstructed.

Turning now to FIG. 16, in accord with a further embodiment of the invention, a unitary (one-piece) anterior spinal plate is 110 shown sized to extend across from one vertebra 52 across a disc space 53 provided with a disc replacement device 94, and to another vertebra 54 of the spine. The plate 110 may be longer to extend across additional vertebral levels. At each level at which the plate 110 is to be attached to the anterior portion of the spine, the plate includes a concavity in the AP dimension along 112, 114 for the vertebra 52 and along 116, 118 for the vertebra 54. Also at each level, in a cephalo-caudal (SI) dimension, a convexity is provided between peripheral portions of the plate, i.e., between 120 and 122 to further accommodate the anatomy of the lateral anterior vertebral surface. Further, at each level, a concave recess 124, 126 is provide at a central portion in the SI dimension and running transverse to the plate in the AP dimension. Each recess 124, 126 defines a space between the plate 110 and the vertebral surface 72 of the respective vertebrae 52, 54 which offsets the lower surface of the plate relative to the segmental vessels 70. Such offset prevents or limits contact of the plate against the vessels and thereby ensures that the segmental vessels remain intact and unobstructed.

There have been described and illustrated herein several embodiments of an anterior vertebral plate, systems utilizing such plate, and methods of implanting such plates and systems. While particular embodiments of the invention have been described, it is not intended that the invention be limited thereto, as it is intended that the invention be as broad in scope as the art will allow and that the specification be read likewise. Thus, while particular plates and systems comprising the plates have been disclosed, it is appreciated that kits of the necessary and desired implants (by way of example and not limitation, plates of multiple sizes, screws, rods of multiple lengths, bridge elements, implant maneuvering tools, bone removal tools, etc.) can be provided to construct the systems in accord with the invention. It will therefore be appreciated by those skilled in the art that yet other modifications could be made to the provided invention without deviating from its spirit and scope as claimed.

What is claimed is:

1. A vertebral plate for placement on an anterior portion of a vertebra having an anterior-posterior dimension and an cephalo-caudal dimension, comprising:
a rigid plate element including a lower bone contacting surface and cephalad and caudal peripheral portions and sized to fit completely on an anterior lateral portion of a vertebra,
the lower bone contacting surface provided with a concavity in an anterior-posterior dimension, and a convexity provided between peripheral portions of the plate, such convexity extending within the cephalo-caudal dimension.

2. A vertebral plate according to claim 1, wherein:
the convexity is defined by the peripheral portions being angled relative to each other.

3. A vertebral plate according to claim 2, wherein:
the angle is between 180° and 240°.

4. A vertebral plate according to claim 2, wherein:
the angle is between 180° and 220°.

5. A vertebral plate according to claim 1, wherein:
the convexity is defined by a curve.

6. A vertebral plate according to claim 1, wherein:
the lower bone contacting surface of the plate defines a recess at a central portion in the cephalo-caudal dimension of the plate and running across the plate in the anterior-posterior dimension.

7. A vertebral plate according to claim 6, wherein:
the convexity is defined by the peripheral portions being angled relative to each other.

8. A vertebral plate according to claim 7, wherein:
the angle is between 180° and 240°.

9. A vertebral plate according to claim 7, wherein:
the angle is between 180° and 220°.

10. A vertebral plate according to claim 6, wherein:
the convexity is defined by a curve.

11. A vertebral plate according to claim 6, wherein:
the plate includes a plurality of screw holes.

12. A vertebral plate according to claim 6, further comprising:
at least one spike provided to the lower bone contacting surface of the plate.

13. A vertebral plate for placement on an anterior portion of a vertebra having an anterior-posterior dimension and a cephalo-caudal dimension, comprising:
a rigid plate element including a lower bone contacting surface contoured to accommodate the anatomy of the lateral anterior vertebra, and including cephalad and caudal peripheral portions, the plate sized to fit completely on an anterior lateral portion of a vertebra,
the lower bone contacting surface of the plate defining a recess at a central portion in the cephalo-caudal dimension of the plate and running across an entirety of the plate in the anterior-posterior dimension.

14. A vertebral plate according to claim 13, wherein:
said lower bone contacting surface including the recess is smoothly contoured.

15. A vertebral plate according to claim 13, wherein:
the recess has a width of 2-10 mm and height of 0.5 to 3 mm.

16. A vertebral plate according to claim 13, wherein:
the recess has a width of 2-5 mm and a height of 0.5 to 2 mm.

17. A vertebral plate according to claim 13, wherein:
the contour of the lower bone contacting surface includes a convexity provided between peripheral portions of the plate, such convexity extending within the cephalo-caudal dimension.

18. A vertebral plate according to claim 17, wherein:
the convexity is defined by the peripheral portions being angled relative to each other.

19. A vertebral plate according to claim 18, wherein:
the angle is between 180° and 240°.

20. A vertebral plate according to claim 18, wherein:
the angle is between 180° and 220°.

21. A vertebral plate for placement on at least one level of an anterior lateral portion of the thoracolumbar spine, the spine defining a cephalo-caudal dimension and an anterior-posterior dimension, said plate comprising:
a rigid plate element provided with a lower bone contacting surface contoured to accommodate the anatomy of each level of the lateral anterior vertebrae for which it is to be coupled, the plate including screw holes for attachment to a body of a vertebra at each such level, and at each such level the plate including cephalad and caudal peripheral portions, the peripheral portions provided with a relative convexity extending within the cephalo-caudal dimension, and
the lower bone contacting surface of the plate defining a plurality of recesses extending transverse the cephalo-caudal dimension and running across the entirety of the width of the plate in the anterior-posterior dimension to accommodate segmental vessels located on respective vertebrae.

22. A vertebral plate according to claim 21, wherein:
the plate has a length to extend across multiple levels.

23. A vertebral plate according to claim 21, wherein:
the lower bone contacting surface is provided with a concavity in the anterior-posterior dimension.

24. A vertebral plate system for placement on an anterior portion of vertebrae, the vertebrae defining an anterior-posterior dimension and a cephalo-caudal dimension, comprising:
a plurality of plates, each plate including a lower bone contacting surface sized to fit completely on an anterior lateral portion of a vertebra, the lower bone contacting surface including cephalad and caudal peripheral portions and a recess at a central portion in the cephalo-caudal dimension of the plate and running across an entirety of the plate in the anterior-posterior dimension;
first means for coupling the plates to a plurality of vertebrae; and
second means for coupling the plates to each other.

25. A vertebral plate system according to claim 24, wherein:
the lower bone contacting surface of each plate is provided with a concavity in the anterior-posterior dimension, and a convexity provided between peripheral portions of the plate, such convexity extending within the cephalo-caudal dimension.

26. A vertebral plate system for placement on an anterior portion of vertebrae, the vertebrae defining an anterior-posterior dimension and a cephalo-caudal dimension, comprising:
a plurality of plates, each plate including a lower bone contacting surface and cephalad and caudal peripheral portions, each plate sized to fit completely on an anterior lateral portion of a vertebra, the lower bone contacting surface of each plate defining a recess at a central portion in the cephalo-caudal dimension of the plate and running across an entirety of the plate in the anterior-posterior dimension;
bone screws adapted to coupled the respective plates to a plurality of respective vertebrae; and
at least one rigid element having a length sized to extend across at least one vertebral level, said plates rigidly coupled relative to said rigid element.

27. A vertebral plate system according to claim 26, wherein:
said recess on each plate is smoothly contoured.

28. A vertebral plate system according to claim 26, wherein:
said recess on each plate has a width of 2-10 mm and height of 0.5 to 3 mm.

29. A vertebral plate system according to claim 26, wherein:
said recess on each plate has a width of 2-5 mm and a height of 0.5 to 2 mm.

30. A vertebral plate system according to claim 26, wherein:
said at least one rigid element is a plate-like bridge element coupled to adjacent plates.

31. A vertebral plate system according to claim 26, wherein:
said at least one stiff element is a plurality of rods extending between said plates.

32. A vertebral plate system according to claim 26, wherein:
the lower bone contacting surface of each plate is provided with a concavity in the anterior-posterior dimension, and a convexity provided between peripheral portions of the plate, such convexity extending within the cephalo-caudal dimension.

33. A vertebral plate system for placement on an anterior portion of vertebrae, the vertebrae defining an anterior-posterior dimension and a cephalo-caudal dimension, comprising:
a plurality of vertebral plates sized to fit completely on an anterior lateral portion of a vertebra, each plate including a lower bone contacting surface including cephalad and caudal peripheral portions, the lower bone contacting surface provided with a concavity in an anterior-posterior dimension and a convexity provided between the caphalad and caudal peripheral portions of the plate, such convexity extending within the cephalo-caudal dimension;
bone screws adapted to coupled the respective plates to a plurality of respective vertebrae; and
at least one rigid element having a length sized to extend across at least one vertebral level, said plates rigidly couplable relative to said rigid element.

34. A vertebral plate system according to claim 33, wherein:
said at least one rigid element is a plate-like bridge element coupled to adjacent plates.

35. A vertebral plate system according to claim 33, wherein:
said at least one stiff element is a plurality of rods extending between said plates.

36. A vertebral bone plate kit for use in treating a condition of the human spine, the spine including vertebrae, the vertebrae defining an anterior-posterior dimension and a cephalo-caudal dimension, comprising:
a first plurality of vertebral plates of a first size sized to fit completely on an anterior lateral portion of a vertebra, each plate including a lower bone contacting surface including cephalad and caudal peripheral portions, the lower bone contacting surface provided with a concavity in an anterior-posterior dimension and a convexity provided between the caphalad and caudal peripheral portions of the plate, such convexity extending within the cephalo-caudal dimension;
a second plurality of vertebral plates of a second size different from said first size, each plate including a lower bone contacting surface including cephalad and caudal peripheral portions, the lower bone contacting surface provided with a concavity in an anterior-posterior dimension and a convexity provided between the caphalad and caudal peripheral portions of the plate, such convexity extending within the cephalo-caudal dimension; and
bone screws adapted to coupled a plurality of said first or second plates to a plurality of vertebrae; and
at least one rigid element having a length sized to extend across at least one vertebral level, said plates first and second plurality of vertebral plates rigidly couplable relative to said rigid element.

37. A kit according to claim 36, wherein:
said lower bone contacting surface of said first plurality of plates includes a recess at a central portion in the cephalo-caudal dimension of the plate and running across an entirety of each said plate in the anterior-posterior dimension, and
said lower bone contacting surface of said second plurality of plates includes a recess at a central portion in the cephalo-caudal dimension of the plate and running across an entirety of each said plate in the anterior-posterior dimension.

38. A vertebral bone plate kit for use in treating a condition of the human spine, the spine including vertebrae, the vertebrae defining an anterior-posterior dimension and a cephalo-caudal dimension, comprising:
a first plurality of vertebral plates of a first size, each plate including a lower bone contacting surface contoured to accommodate the anatomy of the lateral anterior vertebra, each plate of a said first size sized to fit completely on an anterior lateral portion of vertebrae, the lower bone contacting surface of said first plurality of plates includes a recess at a central portion in the cephalo-caudal dimension of the plate and running across an entirety of each said plate in the anterior-posterior dimension;
a second plurality of vertebral plates of a second size different from said first size, each plate including a lower bone contacting surface contoured to accommodate the anatomy of the lateral anterior vertebra, and including cephalad and caudal peripheral portions, each plate of a said second size sized to fit completely on an anterior lateral portion of relatively larger vertebrae, the lower bone contacting surface of said first plurality of plates includes a recess at a central portion in the cephalo-caudal dimension of the plate and running across an entirety of each said plate in the anterior-posterior dimension; and
bone screws adapted to coupled a plurality of said first or second plates to a plurality of vertebrae; and
at least one rigid element having a length sized to extend across at least one vertebral level, said plates first and second plurality of vertebral plates rigidly couplable relative to said rigid element.
